SEQUENCE LISTING (5 sequences)

SEQ ID NO: 1 - DNA, 6458 bp, Artificial sequence
  Plasmid.pcDNA3.1hCD5.6Ala7
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatgcccatggggtctctgcaaccgctggccaccttgtacctgctggggatgctggtcgcttcctgcctcggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 7464 bp, Artificial sequence
  Plasmid pMG10Ala7
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgtaagctgcaataaacaatcattattttcattggatctgtgtgttggttttttgtgtgggcttgggggagggggaggccagaatgactccaagagctacaggaaggcaggtcagagaccccactggacaaacagtggctggactctgcaccataacacacaatcaacaggggagtgagctggatcgagctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcacccgggggatccttcgaacgtagctctagccaccatgcccatggggtctctgcaaccgctggccaccttgtacctgctggggatgctggtcgcttcctgcctcggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 1113 bp, Artificial sequence
  DNA for mutated IL-15/Fc with CD5 leader
atgcccatggggtctctgcaaccgctggccaccttgtacctgctggggatgctggtcgcttcctgcctcggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 370 aa, Artificial sequence
  Amino acid sequence of human CRB-15 with CD5 leader
MPMGSLQPLATLYLLGMLVASCLGNWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 371 aa, Artificial sequence
  Amino acid sequence of murine IL-15/Fc (human mutated IL-15, murine IgG2A) with CD5 leader
MPMGSLQPLATLYLLGMLVASCLGNWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2009/0117618 A1
(43) Pub. Date: May 7, 2009

(54) Expression system for preparing IL-15/FC fusion protein and its use

(76) Inventors: Andreas Herrmann, Koeln (DE); Ingeborg Dreher, Mannheim (DE); Thomas Moll, San Diego, CA (US); Stefanie Zahn, Essen (DE)

Correspondence Address: CLARK & ELBING LLP, 101 FEDERAL STREET, BOSTON, MA, 02110, US

(21) Appl. No.: 10/592,010

(22) PCT Filed: Apr. 13, 2005
(86) PCT No.: PCT/EP05/03888

§ 371 Date: Mar. 6, 2007

(30) Foreign Application Priority Data
Apr. 14, 2004 (EP) 04008881.7

(51) Int. Cl. C07H 21/00; C12N 1/00; C12N 5/00; C12P 21/00; C12N 15/00

(52) U.S. Cl. 435/69.52; 536/24.1; 435/243; 435/325; 435/455

(57) ABSTRACT

The invention relates to an expression system containing one or more nucleic acid(s) comprising at least one nucleic acid for an interleukin 15/Fc (IL-15/Fc) fusion protein, at least one promotor, at least one nucleic acid for a CD5 leader and, where appropriate, at least one nucleic acid for a selectable marker gene; to a nucleic acid comprising the components of the said expression system and to a host cell containing the expression system or the nucleic acid. Furthermore, the invention relates to a process for preparing an IL-15/Fc fusion protein, using the expression system, and to the use of the expression system, the nucleic acid, the host cell or the CD5 leader for expression in host cells.

The invention relates to an expression system which comprises at least one nucleic acid for an interleukin-15/Fc (IL-15/Fc) fusion protein and with the help of which the IL-15/Fc fusion protein may be prepared. Furthermore, the invention relates to a process for preparing an IL-15/Fc fusion protein, using the expression system, and to the use of the expression system, the nucleic acid, the host cell or the CD5 leader for expressing proteins in host cells.

The immune events in mammals are based on a multiplicity of complex cellular and acellular interactions which act like an immune network. The function of many mechanisms within this complex network has been elucidated only in recent times. Cytokines which include the interleukin-15 factor described in 1994 (Grabstein et al., 1994, Science 264: 965-968) play a key part as soluble messengers within the immune network. Interleukin-15 (IL-15) has an influence as immune modulator, growth factor, chemokine and survivor factor on the proliferation, differentiation, activation and survival of cells of the immune system, such as T cells, monocytes/macrophages, NK cells and other IL-15-sensitive cells of the tissue, such as keratinocytes and others. Besides its function as immune moderator, IL-15 also plays a part in the regulation of muscle- and fatty-tissue metabolism.

Typically, IL-15 binds to its effector cells via the heterotrimeric interleukin-15 receptor (IL-15R). IL-15R consists of an α-subunit which binds specifically to IL-15, a β-subunit which is likewise recognized by IL-2 and a γ-subunit which is likewise recognized by further members of the interleukin family, such as IL-2, IL-4, IL-7, IL-9 and IL-15.

IL-15 plays a part in a multiplicity of autoimmune diseases and chronic inflammatory diseases such as, for example, rheumatoid arthritis, psoriasis, multiple sclerosis, Crohn's disease, ulcerative colitis, enterocolitis, pulmonary sarcoidosis or systemic lupus erythematodoses, and also in the immunological rejection of transplanted organs, tissues and cells. IL-15 also plays a part in lymphoid leukaemias.

Interleukin-15 is used therapeutically either according to the agonistic principle, in order to expand lymphocyte populations in cancer patients and in the case of immunodeficiency disorders, or, in the case of disorders with pathological activation of the immune system, according to the antagonistic principle by using agents which block the action of IL-15. These agents may be soluble IL-15-receptor polypeptides, antibodies directed to IL-15 or the IL-15 receptor or they may be fusion proteins having an IL-15 moiety, such as, for example, a fusion protein containing an IL-15 component and an immunoglobulin component (overview in Fehninger and Caligiuri, 2001, Blood 97(1): 14-32). The interleukin-immunglobulin fusion proteins have proved advantageous here.

Recombinant fusion proteins of interleukins and immunoglobulins may be prepared in prokaryotic expression systems. A substantial disadvantage of these expression systems is the lack of glycosylation of the prokaryotically produced proteins, which may impair the functionality and stability of the expressed product and thus limit the medical usability of the expression products. In contrast, production of recombinant fusion proteins of interleukins and immunoglobulins in alternative expression systems such as, for example, mammalian cells, which usually guarantee correct glycosylation, has the problem of a comparatively low expression efficiency (Zheng et al., 1999, J. Immunol. 163: 4041-4048). There exists therefore a need for providing an expression system for eukaryotes, which enables large amounts of recombinant IL-15/Fc fusion proteins to be prepared with sufficient purity.

It was therefore an object of the present invention to provide an improved expression system of this type.

The object was achieved by providing an expression system for preparing an IL-15/Fc fusion protein, containing one or more nucleic acid(s) comprising
a) at least one nucleic acid for an IL-15/Fc fusion protein,
b) at least one promotor and
c) at least one nucleic acid for a CD5 leader,
   the promotor and the nucleic acid for the CD5 leader being functionally linked to the nucleic acid for the IL-15/Fc fusion protein.

It is possible, with the aid of the expression system according to the invention, to prepare IL-15/Fc fusion proteins on a larger scale by means of recombinant DNA technology, for example in eukaryotes. Thus, the present invention enables IL-15/Fc fusion proteins to be prepared for commercial purposes.

Recombinant DNA technology usually means technologies for transferring genetic information, for example to vectors. These vectors enable the genetic information to be processed further, for example by way of introduction into a host, enabling the genetic information to be both multiplied and expressed in a new environment. The genetic information is usually present in the form of nucleic acids, for example in the form of genomic DNA or cDNA, which contains the information for one or more desired gene products in an encoded form. Examples which may act as vectors are plasmids into which nucleic acids such as, for example, cDNA may be integrated in order to be multiplied and, where appropriate, under the control of transcription-regulatory elements such as, for example, promotors, enhancers or silencers, to be expressed in a host cell. Plasmids may contain further elements which influence both the synthesis of the desired expression product and the stability and localization of the latter in the host cell or which enable the plasmid used or expression product to be selected.

The term expression system refers in accordance with the present invention to one or more nucleic acid(s)—where appropriate in combination with further elements which may be necessary for transcription, such as, for example, ribosomes, amino acids and/or tRNAs—it being possible for the expression system to cause expression of the IL-15/Fc fusion protein under suitable conditions, for example in a suitable host cell.

According to a preferred embodiment, the expression system consists of the said one or more nucleic acids.

In order to make expression in host cells possible, the nucleic acid(s) of the expression system may also be part of one or more vector(s) which may be prepared by methods of recombinant DNA technology, which are known to the skilled worker (Sambrook et al. (eds.), 1989, Molecular Cloning: A Laboratory Course Manual. Cold Spring Harbor Press, New York). The skilled worker knows a multiplicity of vectors which may be used in connection with the present invention. Suitable for expression in eukaryotic cells are, for example, the yeast vectors pYES (expression in S. cerevisiae; Invitrogen) and pICZ (expression in P. pastoris; Invitrogen). Baculovirus vectors such as pBacPAK9 (expression in insect cells; Clontech), and also a number of vectors which are used for heterologous expression in mammalian cells, such as Rc/CMV, Rc/RSV, pcDNA and other SV40-derived vectors, into which suitable transcription-regulatory elements may be inserted in addition to the nucleic acid sequences to be expressed, are also usable.

In addition to an origin of replication, which mediates plasmid replication in the chosen host, suitable vectors preferably contain usually selectable marker genes and also recognition sites for restriction endonucleases, which enable nucleic acid fragments to be inserted. The nucleic acid coding for the IL-15/Fc fusion protein may be introduced into the vector via suitable recognition sites for restriction endonucleases.

Viral vector systems which are likewise suitable for the expression system according to the invention comprise, for example, retroviral, adenoviral, adeno-associated viral vectors and also herpes virus or papilloma virus vectors.

The nucleic acid coding for an IL-15/Fc fusion protein is preferably a DNA or RNA, particularly preferably a genomic DNA, a cDNA or combinations thereof.

A nucleic acid for an IL-15/Fc fusion protein codes for an IL-15/Fc fusion protein. An Il-15/Fc fusion protein according to the present invention is a fusion protein which contains two fusion moieties, namely an 11-15 component and an Fc component. Recombinant proteins which contain a fusion moiety of an immunoglobulin in addition to a functional protein are described, for example, in Capon et al. (U.S. Pat. No. 5,428,130).

Preference is given to a fusion protein which consists of an N-terminal mutated or unmutated IL-15 part and a C-terminal Fc part. Such proteins are disclosed, for example, in WO 97/41232 and Kim et al. (1998, J. Immunol. 160:5742-5748).

The IL-15 part of the fusion protein mediates selective binding to the IL-15 receptor (IL-15R) which is expressed on activated T cells, for example. The IL-15 part may therefore be both a naturally occurring IL-15 and a mutant thereof.

In a more preferred embodiment, the IL-15 component is wild-type IL-15. In this connection, the IL-15 may be an IL-15 of any species such as, for example, mice, rats, guinea pigs, rabbits, cattle, goats, sheep, horses, pigs, dogs, cats or monkeys, preferably humans. Included are also different splice variants and naturally occurring variants. Particular preference is given here to nucleic acids of mammals, in particular the human or murine form of the nucleic acids.

IL-15 mutants include IL-15 components which, compared with the naturally occurring IL-15, have a mutation such as, for example, one or more deletions, insertions or substitutions or combinations thereof. The IL-15 variant used, however, must enable the IL-15/Fc fusion protein to bind to IL-15R. This could be checked, for example, in a radioligand binding assay using labelled IL-15 and membranes or cells having IL-15 receptors (Carson W E et al., 1994, J Exp Med., 180(4): 1395-1403).

In a preferred embodiment, the mutant may have an action like IL-15 (IL-15 component with agonist action) and its activity, in comparison with IL-15, may be at the same, a reduced or even an increased level. A test system which may be used for IL-15/Fc fusion proteins having an IL-15 component with agonist action is the stimulation of murine CTLL-2 cell proliferation by the said IL-15 component.

An IL-15 component has agonist action in accordance with the present invention, if the component has at least 10%, preferably at least 25%, more preferably at least 50%, still more preferably 100%, even more preferably 150% and most preferably at least 200% activity.

Activity of an IL-15 component with agonist action means the percentage of stimulation of the response by the IL-15 component in comparison with stimulation by wild-type IL-15 (wild-type IL-15 corresponds to 100% activity). It is possible to use in the tests either the IL-15 component alone or the fusion protein.

For IL-15 components with agonist action, preference is given to conservative amino acid replacements, with a residue being replaced with another one having similar properties. Typical substitutions are substitutions within the group of aliphatic amino acids, within the group of amino acids with aliphatic hydroxyl side chain, within the group of amino acids with acidic radicals, within the group of amino acids with amide derivatives, within the group of amino acids with basic radicals or among the amino acids with aromatic radicals. Typical conservative and semi-conservative substitutions are the following:

| Amino acid | Conservative substitution | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

In another embodiment of the present invention, use is made of IL-15 components with antagonist action. Components of this type inhibit the action of IL-15 or binding of IL-15 to IL-15R, it being possible for the inhibition to be complete or only partial. A test system which may be used for IL-15/Fc fusion proteins which have an IL-15 component with antagonist action is the test system described in WO97/41232 (BAF-BO3 cell proliferation assay). An IL-15 component has antagonist action in accordance with the present invention, if the component inhibits at least 10%, preferably at least 25%, more preferably at least 50% and most preferably at least 95% of the IL-15-mediated action or binding of IL-15 to IL-15R. It is possible to employ in the tests either the IL-15 component alone or the fusion protein.

For IL-15 components with antagonist action, preference is given to non-conservative amino acid replacements, with a residue being replaced with another one having different properties.

Preference is further given to these replacements taking place in regions of the molecule which are responsible for the interaction with IL-15-R or for signal transduction.

In a preferred embodiment, the IL-15 components with antagonist action used are the IL-15 mutants described in WO 97/41232 or an IL-15 component having a mutation at amino acid position 56 (aspartate; AAA21551). Most preference is given to mutants into which point mutations have been introduced at amino acid positions 149 and/or 156 of interleukin-15, replacing glutamine with aspartate in particular (see WO 97/41232). In one embodiment it is also possible to combine the mutations described.

In one embodiment, the mutated IL-15 part of the fusion protein is at least 65%, preferably at least 70%, more preferably at least 85%, still more preferably at least 95% and most preferably at least 99%, identical to the wild-type IL-15, preferably to a human wild-type IL-15 (e.g. database of the National Center for Biotechnology Information, accession number AAA21551), or else other naturally occurring variants (e.g. the variants with accession numbers CAA63914 and CAA71044 of the database of the National Center for Biotechnology Information).

The second functional unit of the IL-15/Fc fusion protein is an Fc component. The Fc part means the constant (c=constant) fragment of immunoglobulins, which can be prepared by papain cleavage and whose amino acid sequence is highly conserved. The Fc fragment is the antibody fragment which usually does not bind any antigens. An Fc part according to the present invention means preferably also an immunoglobulin fragment as defined above which, besides the hinge region, in addition also comprises the constant domains CH2 and CH3.

The Fc component is derived from the Fc part of any antibody, for example of an IgA, IgD, IgG, IgE or IgM, preferably of an IgM or an IgG, more preferably from an Fc part of the subclasses IgG1, IgG2, IgG3 and IgG4.

In a particular embodiment of the invention, the Fc part of the fusion protein is an Fc fragment of an immunoglobulin G (IgG), which lacks the light chains and heavy chains of the IgG variable region. Examples of IgGs which may be used are IgG1, IgG2, IgG2a, IgG2b, IgG3 and IgG4. Preference is given to human or murine IgG1.

It is possible to use for the present invention the entire Fc part of the antibody or only a part thereof. However, the said part of the Fc part should be designed preferably in such a way that the 11-15/Fc fusion protein has a longer half life of circulation in the blood than the IL-15 component without immunoglobulin component. This may be tested by administering to, for example injecting into the bloodstream of, one or more experimental animals the fusion protein and the IL-15 component and comparing the halflifes of circulation in the blood. A longer halflife is indicated by an increase in the halflife by at least 10%, more preferably at least 20%, still more preferably at least 50% and most preferably at least 100%.

The Fc part may also be a Fc part having at least one mutation. The mutated Fc may be mutated in the manner described above for the IL-15 part.

In one embodiment, the mutated Fc part of the fusion protein is at least 65%, preferably at least 70%, more preferably at least 85%, still more preferably at least 95% and most preferably at least 99%, identical to the Fc part of a murine or human wild-type immunoglobulin, preferably to the human IgG 1-Fc or as naturally occurring variants.

In a preferred embodiment of the invention, the Fc moiety of the fusion protein is in the native form or has conservative amino acid replacements and contains intact FcR- and/or complement-binding sites. The Fc moiety of the fusion protein may mediate both activation of the complement system and binding to Fc receptor-expressing cells and thus results in the depletion of the cells recognized by the IL-15 moiety of the fusion protein. The introduction of mutations, in particular of non-conservative amino acid replacements, at the amino acid positions which mediate complement activation and Fc-receptor binding makes it possible to switch off these functions. Examples of these mutations are those of the binding site for the Fc receptor (FcR) or the complement-binding sites (at amino acid positions 214, 356, 358 and/or 435 in the native human IgG1 or Leu 235, Glu 318, Lys 320 and/or Lys 322 in the native murine IgG2A). The replacement of amino acids in these positions usually results in a loss of the lytic and complement-activating function of the Fc moiety (WO 97/41232).

Still further preference is given to an embodiment in which the amino acid cysteine in position 4 of the hinge region of the human Fc moiety, more preferably of the human IgG1 (position 167 of human IgG1), has been replaced with alanine, for example in order to prevent intermolecular bridging and thus aggregation of the expressed IL-15/Fc fusion protein.

In another preferred embodiment the Fc part is the Fc part of the human immunoglobulin IgG1 or of the murine immunoglobulin IgG2A, which, in addition to the hinge region, comprises the heavy-chain regions CH2 and CH3.

In the IL-15/Fc fusion protein, the IL-15 component is fused to the immunoglobulin component either directly or via a linker. The linker consists preferably of no more than 25 amino acids, more is preferably of no more than 15 amino acids, still more preferably of no more than 10 amino acids and most preferably of 1, 2, 3, 4 or 5 amino acids.

In yet another preferred embodiment, a human nucleic acid coding for an interleukin is combined with either a likewise human nucleic acid coding for an Fc or an Fc-encoding nucleic acid of another species such as, for example, mice or rats. For example, a human nucleic acid coding for IL-15 may be combined with a likewise human nucleic acid coding for IgG1, with a murine nucleic acid coding for IgG2A or with a nucleic acid coding for IgG2B from rats. Further possible combinations of nucleic acids will be appreciated by the skilled worker.

The most preferred nucleic acid for an IL-15/Fc fusion protein is the sequence of positions 979 to 2014 of SEQ ID No. 1, that of positions 1985 to 3020 of SEQ ID No. 2 or SEQ ID No. 3 or a nucleic acid coding for the polypeptides of SEQ ID No. 4 or SEQ ID No. 5. The most preferred vector comprising a nucleic acid for an IL-15/Fc fusion protein is a vector of SEQ ID No. 1 or SEQ ID No. 2.

However, the term “nucleic acid for an IL-15/Fc fusion protein” also comprises a nucleic acid whose sequence is at least approx. 60%, preferably approx. 75%, particularly preferably approx. 90% and in particular approx. 95%, identical to the nucleotide sequence indicated in SEQ. ID No. 3 or to a nucleotide sequence coding for the polypeptides of SEQ ID No. 4 or SEQ ID No. 5, the corresponding IL-15/Fc fusion proteins binding to IL-15R and having an increased halflife in the blood compared to the corresponding IL-15/Fc fusion protein without immunoglobulin component (for test systems, see above).

The term “vector comprising a nucleic acid for an IL-15/Fc fusion protein” also comprises a nucleic acid whose sequence is at least approx. 60%, preferably approx. 75%, particularly preferably approx. 90% and in particular approx. 95%, identical to the nucleotide sequences indicated in SEQ. ID No. 1 and SEQ ID No. 2, the corresponding IL-15/Fc fusion proteins binding to IL-15R and having an increased halflife in the blood compared to the corresponding IL-15/Fc fusion protein without immunoglobulin component (for test systems, see above).

The expression system furthermore comprises a promotor. The promotor and its functions are known to the skilled worker. The promotor may be derived from viruses, bacteria or eukaryotes, for example. The promotor may control transcription of the gene to be expressed constitutively or may be inducible and thus make possible a specific regulation of gene expression. The promotor may furthermore be cell- or tissue-specific, i.e. limit expression of the gene product to particular cell types. Promotors having these properties are known to the skilled worker. Promotors which are particularly suitable for controlling expression in a host cell are, for example, the ADH2 promotor for expression in yeast, or the polyhedrin promotor for expression in insect cells. Promotors which mediate strong expression of a gene product in mammalian cells are, for example, viral promotors of viral genes such as the RSV (Rous sarcoma virus) promotor, the SV40 (Simian virus 40) promotor and the CMVi/e (cytomegalovirus immediate early polypeptide) promotor. In connection with the present invention, preference is given to the CMV promotor. Included are also mutations in the CMV promotor, the mutated sequence being preferably 95%, more preferably 99%, homologous to the naturally occurring CMV promotor (Kouzarides et al., 1983, Mol. Biol. Med. 1(1): 47-58) and/or the activity of the mutant, in comparison with the wild-type promotor, being preferably from 90 to 110%, more preferably from 95 to 105%.

In addition, the transcription-regulatory region may, in particular when the CMV promotor is used, contain one or more introns, preferably intron A (Chapman et al., 1991, Nucleic Acids Res. 19(14): 3979-3986). This embodiment has the advantage that it is possible to achieve particularly high amounts of IL-15/Fc fusion proteins, for example by presenting suitable binding sites for transcription factors. Also included are mutations in intron A, the mutated sequence being preferably 80%, more preferably 90% and still more preferably 95%, homologous to a naturally occurring intron, in particular intron A (Chapman et al., 1991, Nucleic Acids Res. 19(14): 3979-3986), and/or the activity of the mutants, compared to the wild-type intron, in particular intron A, being preferably from 90 to 110%, more preferably from 95 to 105%.

Another element of the expression system according to the invention is a nucleic acid for a CD5 leader, i.e. for the secretory signal sequence of the CD5 lymphocyte antigen (Jones et al., 1986, Nature 323 (6086): 346-349). This secretory signal sequence mediates secretion of the expression product into the culture medium of the host cell. The nucleic acid for the CD5 leader and the IL-15/Fc fusion protein are arranged in the expression system such that the leader is able to mediate secretion of the fusion protein. After transcription and translation, the CD5 leader is preferably located in the expression product carboxy-terminally of the fusion protein but may equally preferably also be located amino-terminally of the fusion protein.

Surprisingly, the CD5 leader was shown to mediate in CHO cells 200 to 300 times higher secretion of the expression product into the cell culture medium than comparable signal sequences (see example 2, FIG. 8). Also included are mutations in the CD5 leader, the mutated sequence being preferably 80%, more preferably 90% and still more preferably 95%, homologous to the naturally occurring CD5 leader (Jones et al., 1986, Nature 323 (6086): 346-349) and/or the activity of the mutant, compared to the wild-type CD5 leader, being preferably from 80 to 120%, more preferably from 90 to 110% and still more preferably from 95 to 105%.

In the expression system, the promotor and the nucleic acid for the CD5 leader are functionally linked to the nucleic acid for the IL-15/Fc fusion protein. Functionally linked means that the promotor and the nucleic acid for the leader are arranged, with respect to the nucleic acid for the fusion protein, in such a way that they can exert their function. The function of the promotor is to regulate expression of the fusion protein. If both are located on one nucleic acid, the promotor is usually 5′, or else 3′, of the fusion protein. The function of the leader is to mediate secretion of the fusion protein. If the nucleic acid for the leader and the fusion protein are located on one nucleic acid, the leader usually flanks the fusion protein. “Functionally linked” preferably means that the promotor and the CD5 leader are arranged, in relation to the fusion protein, such that the promotor regulates expression of the fusion protein and the CD5 leader causes secretion of the fusion protein.

In a preferred embodiment, the expression system additionally contains at least one nucleic acid for a selectable marker gene which enables, for example, the host cell transfected with the expression system to be selected over non-transfected cells. Examples of marker genes are resistance-mediating genes which are employed in combination with an antibiotic. The said gene is inserted, for example, into an expression vector and used together with an antibiotic which is applied to the appropriately transfected host cell. Known examples of antibiotics used for selecting eukaryotic host cells are ampicillin, kanamycin, zeocin and, in a preferred embodiment of the invention, neomycin, all of which enable host cells to be selected by expression of the corresponding resistance-mediating gene. The skilled worker knows other marker genes, with, for example, the selective genes tk or DHFR being combined with an application of the corresponding selecting agents such as HAT or aminopterin and methotrexate. Other suitable selectable marker genes such as, for example, the gene of green fluorescent protein from A. Victoria and variants thereof, allow a host cell transfected with the expression vector to be optically selected without being treated with selecting agents.

Preference is given to using the gene coding for the enzyme tryptophan synthetase as selectable marker gene, the corresponding expression plasmid being introduced into a tryptophan synthetase-deficient host cell for selection and expression.

In a further preferred embodiment, the expression system also comprises at least one nucleic acid of a polyadenylation signal which usually, besides terminating transcription, also influences the stability of RNA transcripts. Examples thereof are the polyadenylation sequences from SV40, from the β-globin gene or, in a preferred embodiment, from the bovine growth hormone gene BGH (EP 173552). The nucleic acid of the polyadenylation signal is part of the expression system in such a way that it is capable of improving expression of the fusion protein or its stability. It is usually linked to the nucleic acid for the fusion protein so that the transcription product comprises the IL-15/Fc fusion protein-encoding nucleic acid and the polyadenylation signal.

In yet another embodiment, for example for transcription and/or translation in a cell-free system, the expression system contains, in addition to the above-mentioned components, components which are required for expression. Examples of possible components of this type are transcription factors, enzymes (e.g. peptidyl transferase, aminoacyl-tRNS synthetase and RNA polymerases) and other cellular proteins (e.g. eIF4E, eFE1 and eEF2) and also further auxiliary substances (ATP, GTP and magnesium ions), preferably tRNAs, amino acids and/or ribosomes.

In a preferred embodiment of the invention, the expression system comprises only one nucleic acid which contains the components a) to c) and, where appropriate, d), all of which are as defined above.

In a highly preferred embodiment of the invention, the expression system contains a nucleic acid of any of the sequences SEQ ID No. 1, SEQ ID No. 2 and SEQ ID No. 3 or a nucleic acid coding for a polypeptide of SEQ ID No. 4 or SEQ ID No. 5. Also comprised, however, is a nucleic acid whose sequence is at least approx. 60%, preferably approx. 75%, particularly preferably approx. 90% and in particular approx. 95%, identical to one of the nucleotide sequences indicated in SEQ ID No. 1, SEQ ID No. 2 and SEQ. ID No. 3 or to a nucleotide sequence coding for a polypeptide of SEQ ID No. 4 or SEQ ID No. 5, the corresponding IL-15/Fc fusion proteins binding to IL-15R and having an increased halflife in the blood compared to the corresponding IL-5/Fc fusion protein without immunoglobulin component (for test systems, see above).

In a most preferred embodiment, the expression system comprises a nucleic acid on which the following components are arranged from 5′ to 3′: CMV promotor and, where appropriate, followed by intron A, CD5 leader, IL-15/Fc fusion protein, in particular consisting of an IL-15 having point mutations at amino acid positions 149 and/or 156 of IL-15, replacing glutamine with aspartate (see WO 97/41232), and an Fc part of the human IgG1, in which the amino acid cysteine in position 4 of the hinge region has been replaced with alanine, where appropriate a polyadenylation signal and, where appropriate, at least one marker gene. The marker gene, in particular, may also be arranged on a second nucleic acid. Thus, such a nucleic acid (with or without marker gene) is also a preferred embodiment of the nucleic acid according to the invention.

The present invention further relates to a nucleic acid which comprises the IL-15/Fc fusion protein, the promotor, the CD5 leader, where appropriate the selectable marker gene and, where appropriate, the polyadenylation signal, with all components being as described above. In a preferred embodiment, the nucleic acid contains the sequence of SEQ ID No. 1, SEQ ID No. 2 or 3 or a nucleic acid coding for a polypeptide of SEQ ID No. 4 or SEQ ID No. 5. Also comprised, however, is a nucleic acid whose sequence is at least approx. 60%, preferably approx. 75%, particularly preferably approx. 90% and in particular approx. 95%, identical to one of the nucleotide sequences indicated in SEQ ID No. 1, SEQ ID No. 2 and SEQ. ID No. 3 or to a nucleotide sequence coding for a polypeptide of SEQ ID No. 4 or SEQ ID No. 5, the corresponding IL-15/Fc fusion proteins binding to IL-15R and having an increased halflife in the blood compared to the corresponding IL-15/Fc fusion protein without immunoglobulin component (for test systems, see above).

The invention further relates to a host cell which contains an expression system according to the invention or a nucleic acid according to the invention.

Host cells which may be used are eukaryotic cells such as yeast cells (e.g. S. cerevisiae, P. pastoris), insect cells (e.g. Sf9) or mammalian cells. Examples of mammalian cells of this type are the human embryonic kidney cell line HEK-293, the CHO cell line, prepared from Chinese hamster ovary cells, and its derivatives such as, for example, CHO-K1 and CHO-DHFR, the cell lines BHK, NIH 3T3, HeLa, COS-1, COS-7 and NS.1. The host cell is preferably a mammalian cell, more preferably a CHO cell or its derivatives, most preferably a CHO-K1 cell line.

In a preferred embodiment, the host cells are those cells which have been stably transfected with the nucleic acid(s) of the expression system. In the case of stably transfected cells, the expression system is incorporated into the genome of the target cell and remains in the genome in a stable manner. In contrast to transient transfection, the transferred gene is here not only not degraded but doubled with each cell division and passed onto the daughter cells. The latter thus retain the ability to prepare the desired protein over a long period of time.

Processes for preparing transfected, in particular stably transfected, cells are known to the skilled worker. The host cell may be transformed, for example, by means of electroporation in which permeabilization of the cell membrane, due to briefly applying an electric field, allows nucleic acids to be taken up into the cell, or by way of transfection or infection with a viral vector. Besides transient expression of the recombinant protein, the expression system used may also allow clonal selection of the transfected host cells so that it is possible to select clonal cell lines having a suitable expression efficiency.

In a preferred embodiment, the host cell is a eukaryotic mammalian cell which contains at least one nucleic acid according to SEQ ID No. 1, SEQ ID No. 2 or SEQ ID No. 3 or a nucleic acid coding for the polypeptides of SEQ ID No. 4 or SEQ ID No. 5. Also comprised are the nucleic acids whose sequence is at least approx. 60%, preferably approx. 75%, particularly preferably approx. 90% and in particular approx. 95%, identical to the sequences mentioned, the corresponding IL-15/Fc fusion proteins binding to IL-15R and having an increased halflife in the blood compared to the corresponding IL-15/Fc fusion protein without immunoglobulin component (for test systems, see above).

In a particularly preferred embodiment, the host cell according to the invention is a cell of the CHO-K1 line (subclone of Chinese hamster ovary cells), stably transfected with at least one nucleic acid according to SEQ ID No. 1, SEQ ID No. 2 and/or SEQ ID No. 3 or a nucleic acid coding for the polypeptides of SEQ ID No. 4 or SEQ ID No. 5. Also comprised are the nucleic acids whose sequence is at least approx. 60%, preferably approx. 75%, particularly preferably approx. 90% and in particular approx. 95%, identical to the sequences mentioned, the corresponding IL-15/Fc fusion proteins binding to IL-15R and having an increased halflife in the blood compared to the corresponding IL-15/Fc fusion protein without immunoglobulin component (for test systems, see above).

The invention further relates to a process for preparing an IL-15/Fc fusion protein as defined above, comprising
a. providing a host cell as described above,
b. culturing the host cell,
c. selecting, where appropriate, and
d. isolating the expressed IL-15/Fc fusion protein.

Examples of host cells which may be used are the cells described above. The cell is preferably a mammalian cell, more preferably a CHO cell or derivatives thereof, most preferably a CHO-K1 cell line. A suitable host cell may be transfected with the nucleic acids coding for the IL-15/Fc fusion protein by standard methods (Sambrook et al., 1989, supra).

The transfected host cells may be both adherent cells and a suspension culture. The host cells used are preferably present in a suspension culture, avoiding a reduction in expression efficiency during adaptation of adherent cells to suspension cells.

Primary cells and cell lines may be cultured by standard methods (Freshney, 1993, Animal Cell Culture: A practical approach, John Wiley & Sons, Inc.) in suitable nutrient media under fermentation conditions which have been adjusted to the requirements of the host cells used in each case, with respect to salt concentration, pH, vitamins, trace elements, selecting agents, temperature, aeration, etc., and which enable the desired expression product to be expressed optimally. Advantageously, use is made of nutrient media which are free of serum or foreign proteins and which guarantee a relatively high purity of the expression product.

Selection, in particular clonal selection, means a process in which host cells with desired properties are propagated by step-by-step thinning-out. The process of clonal selection preferably selects those host cell clones which guarantee a sufficient level of expression and/or a pattern of high glycosylation and a high state of sialylation of the expression product. Glycosylation pattern and state of sialylation influence, inter alia, the halflife, biodistribution, immunogeneity and purification behaviour of the expression product. Suitable processes for determining the glycosylation pattern and sialic acid state are known to the skilled worker and comprise, inter alia, combined enzymic cleavages using IEF (isoelectric focussing) and also HPAEC-PAD (High-performance anion-exchange chromatography with pulsed amperometric detection).

The process of the invention furthermore comprises isolating the heterologously expressed IL-15/Fc fusion proteins from the host cells or, in a preferred embodiment, from the culture medium of the host cells. Recombinant polypeptides may be isolated and, where appropriate, purified according to methods known to the skilled worker, which comprise, for example, cell lysis, differential centrifugation, precipitation, gel filtration, affinity chromatography, ion-exchange chromatography, HPLC reverse-phase chromatography, etc. One example of a suitable method for purifying recombinant proteins is affinity chromatography in which an insoluble matrix can bind a ligand due to chemical treatment. A useful ligand may be any molecule having an active chemical group capable of binding to the matrix. The ligand is usually chosen so as to be able to bind to the polypeptide to be purified in a reversible form. The molecule to be purified is applied in the pre-purified culture medium of the host cells to the matrix under conditions which favour binding of the molecule to the ligand, with unbound molecules being removed from the culture medium by a subsequent washing step. The polypeptide to be purified may be eluted by applying a solution which detaches the polypeptide binding to the ligand. Another suitable process is anion-exchange chromatography in which the polypeptides to be purified may bind to the matrix via an excess of positive or negative charge. In a preferred embodiment of the process according to the invention, the expression products are first purified by removing the cell culture medium from the host cells, and this may be followed by a centrifugation and/or filtration step to remove cell debris. In a preferred embodiment, the recombinant IL-15/Fc fusion proteins are purified from the pre-purified culture medium of the host cells by means of a combination of protein-A affinity chromatography and anion-exchange chromatography, which is followed by gel filtration, where appropriate. The expression products obtained in this way may subsequently be characterized with respect to amount, identity and purity by means of methods known to the skilled worker, such as BCA, optical density determination, SDS PAGE, Western Blot, ELISA, amino acid analyzis, amino-terminal sequencing, fingerprinting (MALDI), molecular weight determination (HPLC-ESI), etc.

A particularly preferred process for purifying IL-15/Fc from a composition comprises the following steps:
a) applying the composition to an affinity chromatography column and eluting a first IL-15/Fc eluate from the column;
b) applying the eluate of step a) to an anion-exchange chromatography column and eluting a second Il-15/Fc eluate from the column; and
c) applying the eluate of step b) to a gel filtration column and eluting a third IL-15/Fc eluate from the column.

In a preferred embodiment, the process according to the invention enables an IL-15/Fc fusion protein to be prepared in an amount of at least 10 pg/(cell×day), more preferably of at least 15 pg/(cell×day).

In a further preferred embodiment, the protein after purification is at least 90%, more preferably at least 95% and most preferably at least 99%, pure.

The present invention further relates to the use of an expression system, nucleic acid or host cell as defined above for preparing an IL-15-Fc fusion protein, the use being carried out as described above.

The present invention still further relates to the use of a CD5 leader, as defined above, for expressing a protein in CHO cells and their derivatives, in particular CHO-K1 cells. Surprisingly, expression of the protein or its release into the cell culture supernatant was shown to be 200 to 300 times higher when the CD5 leader is used, compared to expression without leader. In addition, the CD5 leader was shown to be distinctly superior to other leaders in these cells (see example 2, FIG. 8). The protein may be any protein. In a preferred embodiment, expression of the protein is regulated by a CMV promotor, in particular in combination with intron A.

The invention is intended to be illustrated by the following examples and figures, without being limited thereto.

### DESCRIPTION OF THE FIGURES

FIG. 1 depicts a map of the pcDNA3.1hCD5.6Ala7 expression construct.

FIGS. 2-3 depict the sequence of the pcDNA3.1hCD5.6Ala7 expression construct (SEQ ID No. 1).

FIG. 4 depicts a map of the pMG10Ala7 expression construct.

FIGS. 5-6 depict the sequence of the pMG10Ala7 expression construct (SEQ ID No. 2).

FIG. 7A depicts the nucleic acid sequence of the human mutated IL-15/Fc with CD5 leader (SEQ ID No. 3).

FIG. 7B depicts the amino acid sequence of the human mutated IL-15/Fc with CD5 leader (SEQ ID No. 4).

FIG. 7C depicts the amino acid sequence of the human mutated IL-15/Fc with CD5 leader (SEQ ID No. 5).

FIG. 8 depicts the IL-15/Fc content in cell culture supernatants of CHO-K1 cells after transfection with the pcDNA3.1hCD5.6Ala7 plasmid which contained the leader sequence indicated in each case.

FIG. 9 depicts the IL-15/Fc content in cell culture supernatants of CHO-K1 cells after transfection with various expression constructs. Each bar represents the average +SEM of duplicate determinations of in each case two independent experiments.
pcDNA3.1 corresponds to the pcDNA3.1 hCD5.6Ala7 vector.
pVS8-Ala7 corresponds to the pSwitch plasmid (Valentis) with the construct for IL-15/Fc construct.
pMG-Ala7 corresponds to the pMG plasmid (Invivogen) with the construct for IL-15/Fc construct.
pCINeo-Ala7 corresponds to the pCI-Neo plasmid (Promega) with the construct for IL-15/Fc construct.

### EXAMPLES

### Example 1

### Production of IL-15/Fc in CHO-K1 Cells

To produce a CHO-K1 producer cell line for IL-15/Fc an expression construct for IL-15/Fc should be formed and optimized with regard to its secretory properties, to the identity/integrity of the fragments which it contains and to suitable resistance genes.

### A) STARTING MATERIALS

A human IL-15/Fc expression construct (mutated IL-15/human Fc) was provided by the Department of Immunology of the “Beth Israel Deaconness Medical Center” (Harvard Medical School, Boston, USA).

The oligonucleotides were obtained from MWG-Biotech (Ebersberg, Germany). The sequences of the relevant signal peptides were obtained from gene libraries.

The restriction enzymes (BglII, XBaI, BamHI, SmaI, BstXI, ApaI), Lipfectamin2000, other molecular-biological reagents (T4-DNA ligase, T4-polynucleotide kinase) and the plasmids pSecTagA, pcDNA3.1 were obtained from Invitrogen (Karlsruhe, Germany) or Amersham-Pharmacia (NheI, Protein A Sepharose Uppsala, Sweden).

Competent E. coli XL10-Gold cells were obtained from Stratagene (La Jolla, USA). The BCA kit (Pierce) was purchased from KMF Laborchemie (Sankt Augustin, Germany).

The plasmid-DNA purification kits (Endofree-Maxi Kit, Endofree-Giga Kit) were from Qiagen (Hilden, Germany).

The antibodies were obtained from BD-Pharmingen (mouse-anti-hIL-15; catalogue number 554712; Heidelberg, Germany) and Dianova (goat-anti-mouse-POD; catalogue number 15-036-003; goat-anti-human-POD; catalogue number 109-036-088; Hamburg, Germany).

### B) METHODS/RESULTS

The starting plasmids contained within the pSecTagA vector backbone the cDNA of a fusion protein comprising a mutated human IL-15 fused to the Fc part (hinge region and CH2, CH3 regions) of human IgG1. The structure of the plasmid corresponds to that described by Kim et al. (J. Immunol., 160: 5742-5748; 1998), except that the Fc part cited in this application is a murine Igγ2a.

The Igk leader which is already present in the pSecTagA vector was used for secretion of the fusion protein by in-frame cloning of the IL-15/Fc part. For this, the intrinsic signal sequence was removed from the native IL-15 sequence. Due to the cloning, however, 10 additional amino acids were introduced between the 3′ end of the Igk-leader sequence and the 5′ end of the IL-15 coding sequence, which were retained in the secreted protein after processing of the protein. In order to remove these unspecific amino acids and to improve the secretory properties of the protein, various leader sequences of other secretory or cell-surface proteins were tested: murine Igk (Coloma et al., J. Immun. Methods 152: 89-104; 1992; accession number X91670), human CD5 (Jones et al., Nature 323: 346-349; 1986; accession number X04391), CD4 (Hodge et al., Hum. Immunol. 30: 99-104; 1991; accession number M35160), MCP-1 (Yoshimura et al. Je. FEBS Lett. 244: 487-493; 1989; accession number M24545) and IL-2 (Taniguchi et al., Nature 302: 305-310; 1983; accession number K02056) (accession numbers are based on the “National Center for Biotechnology Information”). After removing the Igk leader and the additional amino acids, the leader was replaced with the signal peptide sequences mentioned by cloning double-stranded oligonucleotides. The identity was checked by sequencing. Subsequently, the resulting constructs were tested by transient transfection of HEK-293 cells, using Lipfectamin2000. The protein content of the cell culture supernatants of the cells which have been transfected with the various constructs was measured by means of the BCA assay, after a protein-A-Sepharose purification according to the method by Moll and Vestweber (Methods in Molecular Biology, 96: 77-84, 1999). The identity of the protein was checked by means of silver staining of the SDS gel and Western blots against either the Fc or the IL-15 part, in order to ensure the presence of both components of the fusion protein. The CD5 leader gave the best results in the experiments described and was selected for further optimization of the vector.

It was furthermore tested, whether replacing the cDNA of the Fc part with the genomic DNA containing exon/intron structures also contributes to improved protein expression. The presence of introns which have to be removed by the splice apparatus of the nucleus may improve RNA export from the nucleus and also RNA stability. Therefore, the genomic Fc part was linked to the IL-15 cDNA sequence by inserting splice-donor and splice-acceptor sites. The resulting plasmids were likewise modified by various leader sequences and tested as described above. Protein analyzis by Western blot, however, revealed that various undesired splice variants were present so that it was decided to continue using the cDNA form of the Fc part.

Consequently, the resulting plasmid comprises a human CD5 leader and a cDNA-Fc part.

Sequencing of the mutated IL-15/Fc expression construct revealed that the Fc part contained 3 mutations which were already present in the original construct. Two of these mutations related to amino acids at highly conserved positions. The third mutation was a Cys-Ala mutation at position 4 in the hinge region, which was inserted deliberately in order to stop the formation of intra- and intermolecular cysteine bridges.

In order to remove the two undesired mutations while retaining the Cys-Ala mutation, the Fc cDNA was subcloned by means of RT-PCR. The RNA source used was a CHO-K1 cell line transfected with a construct coding for a VCAM-1-Fc fusion protein. The amplified Fc-cDNA fragment was cloned into the CD5-mutIL-15 plasmid, and the Fc part was removed by BamHI/XbaI restriction.

The resulting plasmid was analyzed again on the basis of distinct restriction patterns and by means of subsequent sequencing and referred to as CD5-6Ala7. Since the use of zeocin as DNA-intercalating agent could cause mutations, the expression cassette for IL-15/Fc was removed from the original pSecTagA backbone and cloned into pcDNA3.1 which contains the neomycin-resistance gene under the control of the SV40 promotor. Both strands of the resulting plasmid were sequenced and revealed complete correspondence with the IL-15/Fc expression cassette.

The construct was again tested for its protein expression by means of transient transfection of CHO-K1 cells and Western blot analyzes of the cell culture supernatant. As a positive control, a transfection with the CD5-6Ala7 plasmid was carried out in a parallel experiment.

To this end, the cells were seeded in triplicates at a density of 5×10⁵ cells per well in tissue culture plates with 6 wells. 2 μg of plasmid and 4 μl of Lipofectamin2000, each of which were diluted in 250 μl of Optimem1 medium, were used for transfection. Both solutions were mixed and, after incubation at room temperature for 30 min, the mixture was pipetted into the culture media of the tissue culture plates.

2 days after transfection, the culture medium was removed and analyzed for its IL-15/Fc content by means of a Western blot against the human IL-15 part: 20 μL of the cell culture supernatant were mixed with 5 μl of 5× Laemmli buffer and incubated at 85° C. for 5 min. The samples were then run on a 12% polyacrylamide gel. The gel was then blotted using a semi-dry blotting chamber. The blot was treated with blocking solution containing 5% milk powder in PBS, 0.1% Tween20 overnight. The blot was then incubated with a monoclonal mouse-anti-human-IL-15 antibody in a 1:1000 dilution in blocking solution for 4 hours. After 3 washing steps (10 min PBS, 0.1% Tween20), the blot was incubated with the secondary antibody, goat-anti-mouse peroxidase (dilution 1:5000), at room temperature for another 2 hours. The blot was washed again 3 times and then Lumilight solution was applied dropwise to the blot surface and an X-ray film was exposed to the blot.

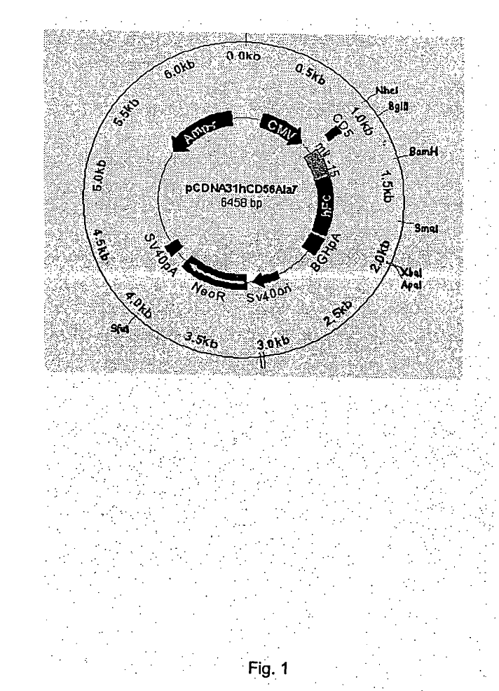

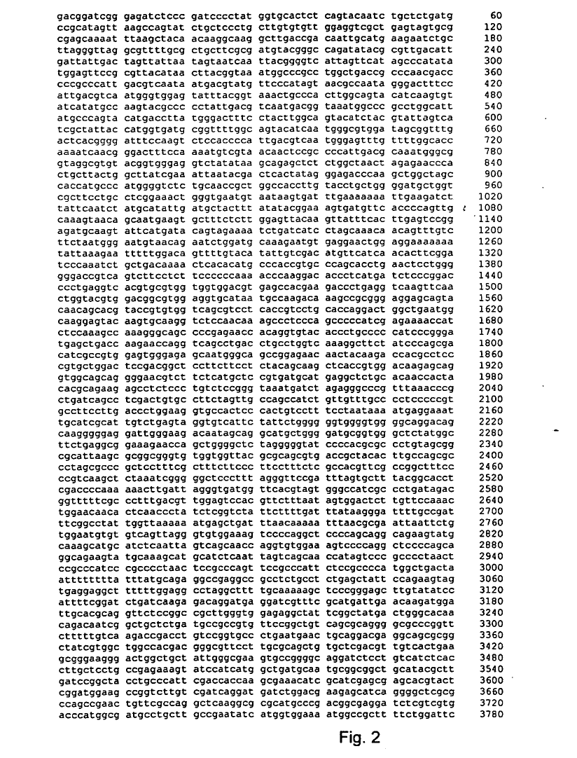

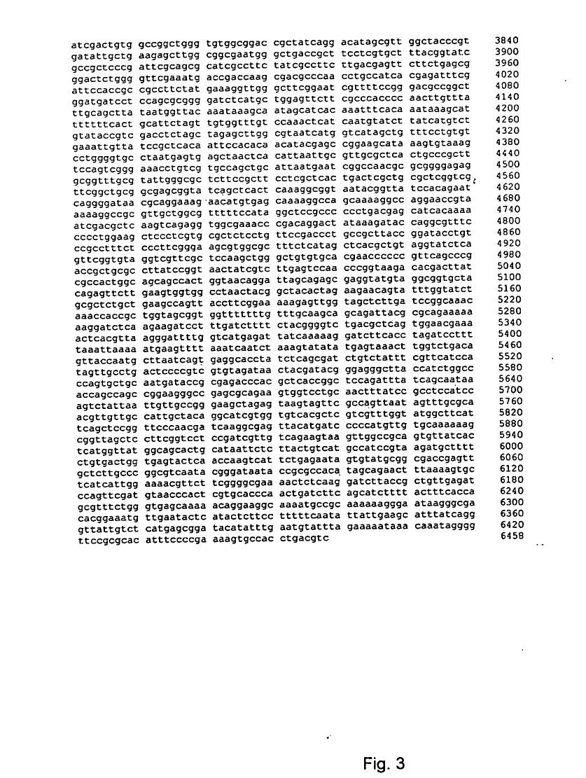

Specific Western blot signals within the range of signals obtained after transfection with CD5-6Ala7 revealed that the cell culture supernatants of all three parallel transfections contained IL-15/Fc as protein. It was therefore shown that the pcDNA3.1hCD5.6Ala7 plasmid (FIGS. 1 to 3) can be used for protein expression in CHO-K1 cells.

### C) CONCLUSIONS

An IL-15/Fc plasmid, pcDNA3.1hCD5.6Ala7, was prepared, which contained an expression cassette containing a CD5 leader with a mutated human IL-15 fused to the cDNA of human IgG1-Fc under the control of the CMV promotor. To select stable eukaryotic cell clones, a neomycin resistance gene was introduced. The plasmid was sequenced and revealed 100% correspondence in the relevant coding regions, with only a slight discrepancy (repeat of 3 base pairs) without any relevance in the vector backbone. The functionality of the construct was checked by transient transfection of CHO-K1 cells.

### Example 2

Transfection of eukaryotic cell lines (e.g. CHO-K1 cells) with a plasmid containing the DNA for the desired product is a standard process for producing therapeutic proteins. Nevertheless, the low productivity levels of the stable cell clones produced in this way are a widely known problem. There are therefore various strategies to increase the productivity of an existing cell line. Apart from the attempt to increase the number of plasmid copies in the cell (e.g. via the methotrexate/DHFR system), it is furthermore possible to modify the expression construct itself. In addition to a strong promotor (e.g. the CMV promotor), introducing an intron possibly results in better RNA stability and better RNA export from the nucleus, which export is carried out by the splice apparatus of the cell. Nevertheless, a test must be carried out as to which combination of intron/transgene is suitable. For this purpose, various introns were combined with the human IL-15-Fc in order to find a combination which increases IL-15-Fc production by CHO-K1 cells.

### A) MATERIALS

The plasmid used as starting plasmid was the pcDNA3.1hCD5.6Ala7 plasmid. It is depicted schematically in FIG. 1. Its sequence is disclosed as SEQ ID No. 1.

The test system used was either CHO-K1 cells (DSM, Braunschweig, Germany, accession number: ACC110) or HEK-293 cells (Qbiogene, Grünberg, Germany, AE80503, QBI-293A). E. coli cells (XL10-Gold, Strategene, La Jolla, USA) were also used. The cells were cultured under standard culturing conditions (5% CO₂, 37° C., humidified atmosphere). The CHO-K1 cells were passaged twice a week at a ratio of 1:20, with the HEK-293 cells being passaged at a ratio of 1:6. The medium used was DMEM-F12+10% FKS+1% PEN/Strep, for the CHO-K1 cells, and DMEM+Glutamax+10% FKS+1% PEN/Strep, for the HEK-293 cells. Optimem1 medium was used for transfection. All media were obtained from by Invitrogen, Karlsruhe, Germany (catalogue numbers 31331-028; 32430-027; 51985-018). The plasmid used was pCl-Neo (Promega) containing a CMV promotor and a chimeric intron, a 5′ splice-donor site of the human beta-globin gene and a 3′ splice-acceptor site of the IgG-heavy chain of the variable region. pMG (Invivogen) is a prolonged CMV promotor containing an intron A from CMV. pSwitch (Valentis) is a synthetic intron, IVS8. Furthermore, the following enzymes and restriction enzymes were used: ApaI, EcoRV, XbaI, NruI, PacI, SmaI, XhoI, T4-DNA ligase, T4-DNA polymerase, alkaline phosphatase from calf intestine. These and other molecular-biological reagents (Lipofectamin2000) were obtained from Invitrogen. NheI was obtained from Amersham-Pharmacia (Uppsala, Sweden) and the plasmid purification kits were obtained from Qiagen, Hilden, Germany. The Expand High Fidelity PCR system (catalogue number 1 732 641) was obtained from Roche, Mannheim, Germany.

### B) METHODS

i) The IL-15/Fc insert of the pcDNA3.1hCD5.6Ala7 plasmid was isolated by way of NheI/ApaI digest. The plasmid was first linearized by ApaI restriction and the 5′-protruding ends were blunted by T4-polymerase treatment. The IL-15/Fc insert was then isolated by subsequent digestion with NheI. The fragment was ligated with pcI Neo which had been digested with NheI and SmaI.
ii) The CMV promotor of pcDNA3.1hCD5.6Ala7 was removed and replaced with the extended CMV promotor with intron A, which was derived from pMG: the pMG plasmid was cleaved with PacI and the protruding ends were blunted by means of T4-polymerase treatment. After a second XbaI treatment, the 1.7 kb fragment obtained in this way, which contained the CMV promotor+intron A, was purified by agarose gel electrophoresis. The CMV promotor was removed from pcDNA3.1hCD5.6Ala7 by means of restriction digestions with NheI and, subsequently, NruI. The resulting fragment was ligated with the pMG-promotor-intron overnight at 4° C.
iii) The IVS8 intron was amplified by means of PCR and cloned between the 3′ end of the CMV promotor and the 5′ end of the IL-15 insert in pcDNA3.1hCD5.6Ala7. The plasmid was linearized by means of NheI restriction digestion and subsequently treated with alkaline phosphatase from calf intestine. The intron was amplified by means of PCR using primers containing XbaI restriction cleavage sites, using the Expand High Fidelity PCR system under the following conditions: the reaction mixture used consisted of 2 μl of dNTPs (Qiagen, Taq core kit, 2 mmol/l each), 25 pmol of primers, 5 μl of 10× buffer, 0.75 μl of High Fidelity Taq polymerase, 1 μl (approximately 15 ng) of pSwitch-XhoI/EcoRV fragment, with water being added to a final volume of 50 μl. The PCR programme (25 cycles) was as follows: 5 min at 95° C., 15 s at 94° C., 30 s at 55° C., 30 s at 72° C., 5 min at 72° C. The PCR product was cleaved with XbaI, eluted from a 0.8%-agarose gel and ligated with the linearized plasmid.

The resulting plasmids were transformed into E. coli XL10 Gold and the plasmids were analyzed by means of miniprep. One clone of each plasmid which exhibited an appropriate restriction pattern was used for subsequent endotoxin-free plasmid preparation.

IL-15/Fc expression was analyzed after transient transfection of HEK-293 or CHO-K1 cells. One day before transfection, the cells were seeded at a density of 5×10⁵ cells per well in cell culture plates with six wells in duplicates. For transfection according to Felgner et al. (Proc. Natl. Acad. Sci. USA, 84:7413-7417; 1987), 2 μg of plasmid and 4 μl of Lipofectamin2000 were diluted in each case in 250 μl of Optimem1 medium. Both solutions were mixed and, after 30 minutes of incubation at room temperature, the mixture was pipetted into the cell culture medium in the cell culture plates. Two days post transfection, the culture medium was removed and its IL-15/Fc content was determined by an ELISA test targeting the Fc part of IL-15/Fc.

### C) RESULTS

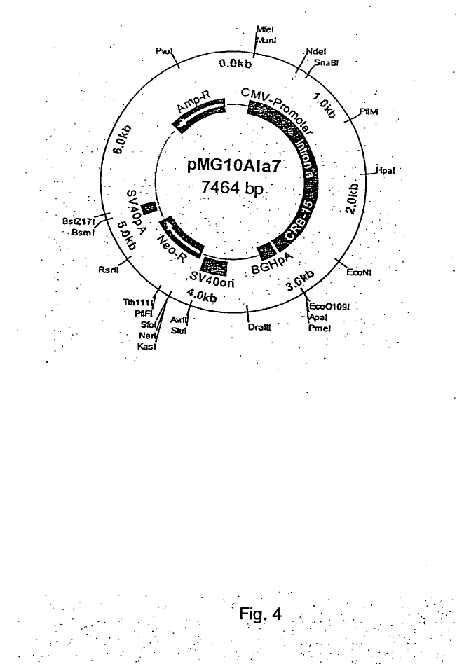

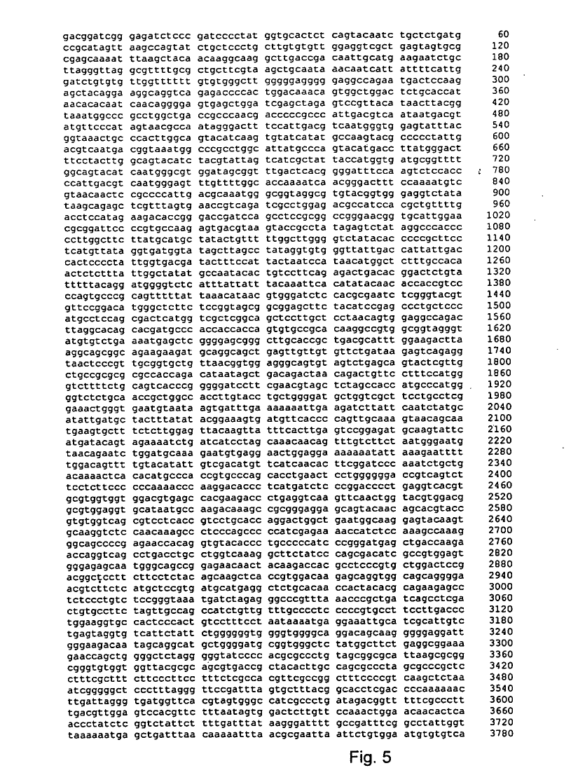

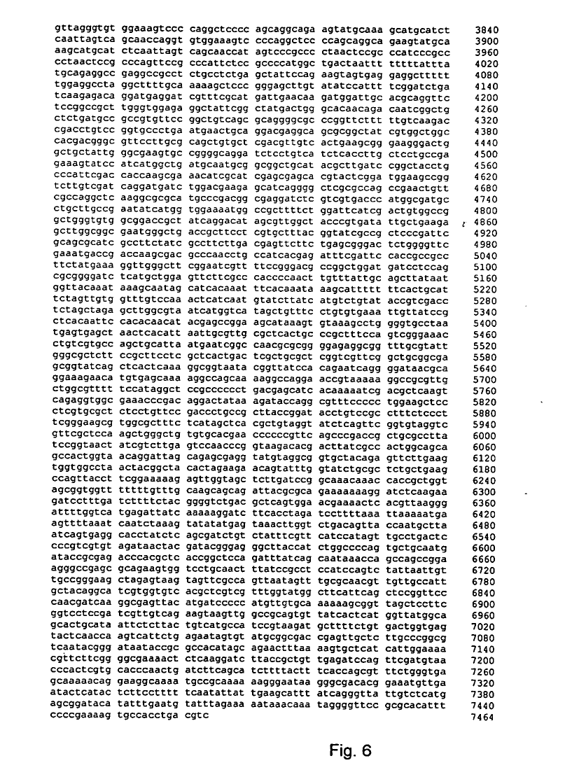

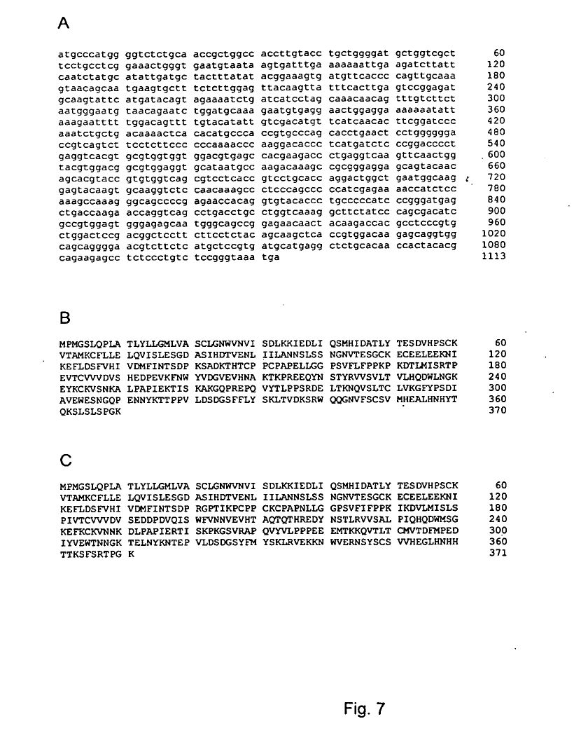

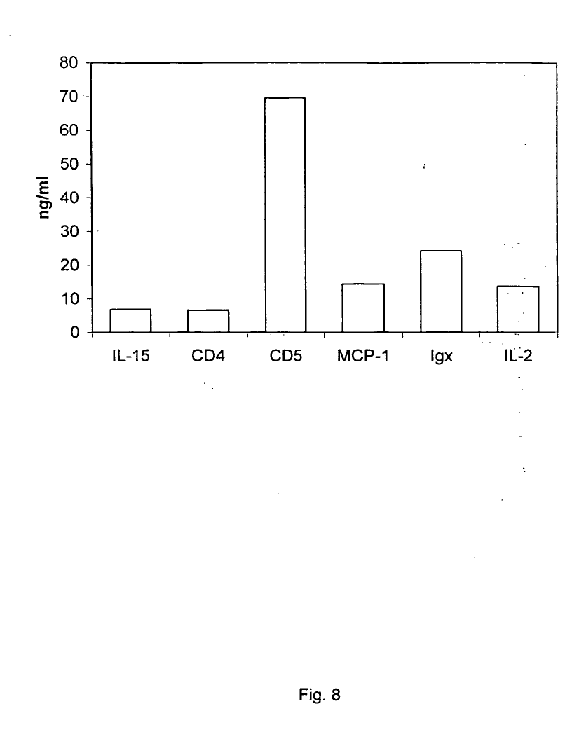

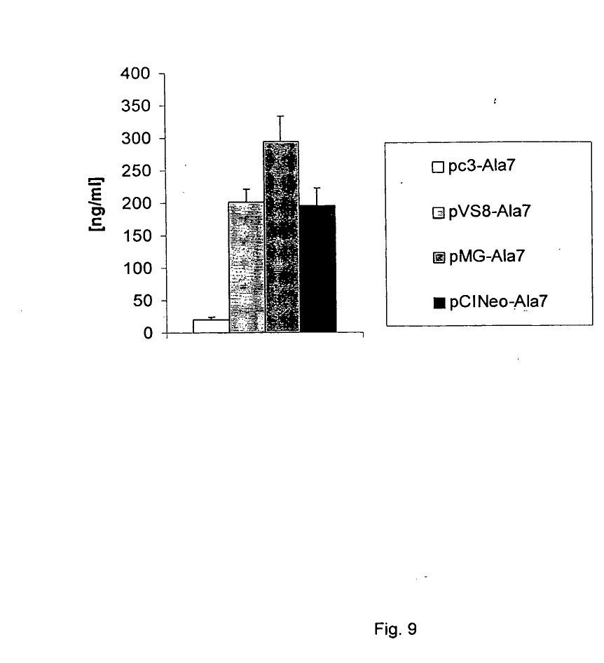

The secretion of IL-15/Fc by HEK-293 cells transfected with various expression constructs was hardly influenced by other vector components. In contrast, expression of IL-15/Fc by CHO-K1 cells had increased by a factor of 200-300 after insertion of an intron into the IL-15/Fc construct. The original construct, pcDNA3.1hCD5.6Ala7, resulted in protein secretion levels which were hardly detectable (below 10 ng/ml), with insertion of an intron resulting in IL-15/Fc levels of approximately 300 ng/ml, after the cells had been transfected with pMG10Ala7 (FIGS. 4 to 6; SEQ ID No. 2). The ELISA data which indicate the IL-15/Fc expression levels in CHO-K1 cells are depicted in FIG. 4. Since the expression levels were highest after transfection with the pMG construct, the latter was chosen for producing a stable CHO-K1 expression cell line.

To this end, the plasmid was first subjected to single-strand sequencing. Both strands of the construct were sequenced in the region which contained the IL-15/Fc cassette, the newly inserted CMV promotor and the intron fragment. The plasmid contained the IL-15/Fc cassette under the control of the CMV promotor. The intron A which was derived from CMV (plasmid MG) was positioned between the promotor and the start of translation. The plasmid contained a BGHpolyA site downstream of the IL-15/Fc fragment; the neomycin-resistant gene was controlled by an SV40 promotor and also contained an SV40polyA site. The plasmid contained an ampicillin-resistance gene for selection and amplification in E. coli.

### D) DISCUSSION AND CONCLUSIONS

In order to increase the protein yield of stable CHO-K1-IL-15/Fc transfectants, the expression plasmid was modified by introducing an intron between the promotor and the IL-15/Fc cassette. The combination intron-transgene-host cell greatly influences protein expression, and therefore it is not possible to predict the intron which is the most effective in increasing IL-15/Fc expression in the two cell types analyzed.

While HEK-293 cells were hardly influenced by introduction of the intron, a large increase in IL-15/Fc secretion was detected in CHO-K1 cells. Expression of the IL-15/Fc protein in CHO-K1 cells increased by more than an order of magnitude in comparison with the original IL-15/Fc expression vector, using a plasmid which contained the CMV promotor and intron A from pMG. The plasmid may be used for producing an IL-15/Fc producer cell line which may be used for producing IL-15/Fc for pre-clinical and clinical studies or else for industrial production of IL-15/Fc.

1. Expression system, containing one or more nucleic acid(s) comprising
a) at least one nucleic acid for an IL-15/Fc fusion protein,
b) at least one promotor and
c) at least one nucleic acid for a CD5 leader,
the promotor and the nucleic acid for the CD5 leader being functionally linked to the nucleic acid for the IL-15/Fc fusion protein.

2. Expression system according to claim 1, in which the promotor is a CMV promotor.

3. Expression system according to claim 1, in which the promotor is part of a transcription-regulating unit which additionally contains an intron.

4. Expression system according to claim 1, in which the Fc part of the fusion protein is an Fc fragment of an immunoglobulin G.

5. Expression system according to claim 1, additionally containing
d) at least one nucleic acid for a selectable marker gene.

6. Expression system according to claim 1, additionally containing at least one nucleic acid for a polyadenylation signal.

7-9. (canceled)

10. Nucleic acid, containing the components a) to c) of claim 1.

11. (canceled)

12. Host cell, containing an expression system according to claim 1 or a nucleic acid according to claim 10.

13-14. (canceled)

15. Process for preparing an IL-15/Fc fusion protein, comprising
a. providing a host cell according to claims 12,
b. culturing the host cell,
c. selecting, where appropriate, and
d. isolating the expressed IL-15/Fc fusion protein.

16-18. (canceled)

19. Method of expressing a protein in a CHO cell or a derivative thereof comprising
a) functionally linking the nucleic acid encoding the protein to the nucleic acid encoding the CD5 leader; and
b) expressing the protein in the CHO cells and or the derivatives thereof.

20. (canceled)

21. The expression system of claim 3, wherein the intron is intron A.
